(19) United States
(12) Patent Application Publication
(10) Pub. No.: US 2016/0213259 A1
(43) Pub. Date: Jul. 28, 2016

(54) OBJECT INFORMATION ACQUIRING APPARATUS

(71) Applicant: CANON KABUSHIKI KAISHA, Tokyo (JP)

(72) Inventor: Koichiro Wanda, Yokohama-shi (JP)

(21) Appl. No.: 14/988,832

(22) Filed: Jan. 6, 2016

(30) Foreign Application Priority Data
Jan. 27, 2015 (JP) 2015-013245

(51) Int. Cl. A61B 5/00

(52) U.S. Cl. CPC A61B 5/0095; A61B 5/708; A61B 2576/00

(57) ABSTRACT

An object information acquiring apparatus is employed which includes elements each receiving a photoacoustic wave from a region of interest of an object and outputting an electric signal, a probe that supports the elements such that directional axes of the elements are gathered, and that holds an acoustic matching member, a scanner that moves the probe, a controller, and a processor that generates image data on the region of interest. The controller performs, on the same region of interest, photoacoustic measurements in which the probe is moved along a curved trajectory, with the elements each allowed to receive a photoacoustic wave, and moves the probe in the same rotating direction as direction of the curved trajectory at a time between the photoacoustic measurements.

### BACKGROUND OF THE INVENTION

1. Field of the Invention

The present invention relates to an object information acquiring apparatus.

2. Description of the Related Art

An effort is being made mainly in the medical field to study optical imaging apparatuses that irradiate an object with light from a light source such as a laser to obtain information on the interior of an object. By way of example, an apparatus using photoacoustic tomography (PAT) has been proposed. The PAT is a technique that involves irradiating an object with light and receiving and analyzing an acoustic wave resulting from absorption, by an object tissue, of the light propagated and diffused through the object, to visualize information on optical characteristics of the interior of the object. Thus, characteristics information such as the distribution of optical characteristic values in the object can be acquired and imaged.

Examples of information related to the optical characteristics include an initial sound pressure distribution and an optical-energy absorption density distribution. Such information is useful for examining the inside of biological tissues and is expected to be utilized for medical diagnoses, for example, localization of a malignant tumor with proliferating newborn blood vessels.

“Photoacoustic angiography of the breast”, Medical Physics, Vol. 37, No. 11, November 2010 discloses a PAT apparatus including a semispherical probe with reception elements arranged in a semicircular form and each receiving and converting the acoustic wave into an electric signal. In the semispherical probe, directions of the reception elements that have high sensitivity are gathered near the center of the sphere to form a high-resolution region. Characteristic information on the high-resolution region is subjected to reduced artifacts than characteristics information on other regions.

Japanese Patent Application Laid-open No. 2012-179348 also discloses a photoacoustic tomography apparatus with a semispherical probe. This apparatus includes a scan apparatus that moves the relative positions of the semi spherical probe and the object in order to enlarge the high-resolution region.

When the semispherical probe is used, an acoustic matching member is needed to match the impedance of the object with the impedance of the reception elements. For example, in Japanese Patent Application Laid-open No. 2012-179348, the space between the object and the reception elements is filled with a liquid such as water.

Patent Literature 1: Japanese Patent Application Laid-open No. 2012-179348

“Photoacoustic angiography of the breast”, Medical Physics, Vol. 37, No. 11, November 2010

### SUMMARY OF THE INVENTION

In the photoacoustic tomography apparatus including the semi spherical probe, matching of the acoustic impedances is achieved by filling the inside of the semisphere with the acoustic matching member. To measure a wide area of the object, measurement may be performed with the probe moved. However, movement of the probe leads to such disturbance as to make the liquid surface of the acoustic matching member wavy. When the liquid surface at the interface between the object and the acoustic matching member is disturbed and mixed with air, a path from the object to the reception element is disrupted, making reception of the acoustic wave difficult. A fluctuation of the liquid surface may make a path of light irradiation unstable. In particular, when the semispherical probe is integrated with a light irradiator, the path of light irradiation may be mixed with air to cause the amount of light and an irradiation light distribution to deviate from expected values.

In “Photoacoustic angiography of the breast”, Medical Physics, Vol. 37, No. 11, November 2010, spiral movement is illustrated as an example of a method for moving the probe. In this moving method, in an XY plane below the object in the vertical direction, the whole probe moves along a spiral trajectory. During the spiral movement, the acoustic matching member rotates in a fixed direction, restraining the liquid surface from being disturbed. However, when the probe performs sharp turnaround or rapid acceleration or deceleration or stops suddenly, the flow of the acoustic matching member is disturbed. This problem occurs when the probe in a stationary state moves at the start of measurement or when the probe stops at the end of the measurement. The problem also occurs when the probe moves from a home position to a measurement start position or when the probe returns from a measurement end position to the home position. The problem also occurs during movement of the probe at the time between photoacoustic measurements if the probe repeats a plurality of spiral movements in the same area.

When the flow of the acoustic matching member is disturbed, mixture of bubbles or formation of air layers may occur near the interface between the acoustic matching member and the object or a holding member installed in tight contact with the object. Moreover, when significant disturbance of the liquid surface results in outflow of the acoustic matching member, reducing water level, the object is exposed over a wide range. As a result, the acoustic matching is insufficient, leading to reduced accuracy of photoacoustic measurement and degraded quality of reconstructed images.

When the liquid surface of the acoustic matching member is disturbed, a certain amount of time is needed for convergence. Thus, when photoacoustic measurements are to be consecutively performed, suppression of disturbance of the liquid surface needs to be maximized. For example, a plurality of photoacoustic measurements with the probe spirally moved may be repeated. In this case, although the liquid surface is stable during each period of spiral movement but is disturbed if rapid acceleration, deceleration, or turnaround occurs when one spiral movement shifts to the next spiral movement.

With the above-described problems in view, it is an object of the present invention to suppress disturbance of the liquid surface of the acoustic matching member held by the probe.

An aspect of the present invention provides an object information acquiring apparatus comprising:

a light source;

a plurality of elements each receiving a photoacoustic wave generated in a region of interest of an object irradiated with light from the light source, and outputting an electric signal;

a probe that supports the plurality of elements such that directional axes of at least some of the plurality of elements are concentrated, and that holds acoustic matching member acoustically matching the object with the plurality of elements;

a scanner that moves the probe;

a controller that controls the light source, the plurality of elements, and the scanner; and

a processor that generates image data on the region of interest using electric signals output by the plurality of elements upon receiving the photoacoustic wave at each position to which the probe is moved by the scanner,

wherein the controller performs, on the same region of interest, a plurality of photoacoustic measurements in which the probe is moved along a curved trajectory in a scan region corresponding to the region of interest, with the plurality of elements each allowed to receive the photoacoustic wave, and moves the probe in the same rotating direction as a rotating direction of the curved trajectory at a time between the photoacoustic measurements, and

the processor generates a plurality of the image data based on the plurality of photoacoustic measurements in the same region of interest.

The aspect of the present invention enables the liquid surface of the acoustic matching member held by the probe to be restrained from being disturbed.

Further features of the present invention will become apparent from the following description of exemplary embodiments with reference to the attached drawings.

### BRIEF DESCRIPTION OF THE DRAWINGS

FIG. 1 is a functional block diagram of a photoacoustic measurer;

FIG. 2 is a diagram of a configuration of an information processor;

FIG. 3 is a flowchart depicting a process executed by an object information acquiring apparatus in connection with a user's operation;

FIGS. 4A, 4B, and 4C are diagrams depicting movement of a probe;

FIG. 5 is a flowchart illustrating a process executed by the photoacoustic measurer; and

FIG. 6 is a flowchart illustrating a process executed by the information processor.

### DESCRIPTION OF THE EMBODIMENTS

Now, with reference to the drawings, a preferred embodiment of the present invention will be described. However, the dimensions, materials, shapes, and relative arrangements of components described below should be modified as needed according to a configuration of and various conditions for an apparatus to which the present invention is applied, and are not intended to limit the scope of the present invention to the descriptions below.

The present invention relates to a technique for generating and acquiring characteristics information on the interior of an object. Therefore, the present embodiment is considered to be an object information acquiring apparatus or a method for controlling the object information acquiring apparatus, or an object information acquiring method and a signal processing method. The present invention is also considered to be a program that allows an information processing apparatus with hardware resources such as a CPU to execute these methods and a storage medium that stores the program.

An object information acquiring apparatus includes an apparatus that utilizes a photoacoustic tomography technique to irradiate an object with light (electromagnetic wave) and to receive an acoustic wave propagating after being generated inside the object or a particular position on a surface of the object in accordance with a photoacoustic effect. Such an object information acquiring apparatus may also be referred to as a photoacoustic imaging apparatus because the apparatus obtains characteristics information on the interior of an object in the form of image data or the like based on photoacoustic measurement.

Characteristics information for the photoacoustic apparatus includes a source distribution of sources of an acoustic wave resulting from light irradiation, an initial sound pressure distribution in the object, or an optical-energy absorption density distribution or an absorption coefficient distribution derived from the initial sound pressure distribution or a concentration distribution of concentrations of substances included in a tissue. A specific example of the characteristics information is a blood component distribution such as an oxyhemoglobin and deoxyhemoglobin concentration distribution or an oxygen saturation distribution determined from the oxyhemoglobin and deoxyhemoglobin concentration distribution, or a distribution of fat, collagen, or moisture. Alternatively, the characteristics information may be distribution information at each position in the object instead of numerical data. That is, object information may be distribution information such as an absorption coefficient distribution or an oxygen saturation distribution.

The acoustic wave as used herein is typically an ultrasonic wave and includes an elastic wave referred to as a sound wave or an acoustic wave. An acoustic wave resulting from a photoacoustic effect is referred to as a photoacoustic wave or light-induced ultrasonic wave. An electric signal into which an acoustic wave is converted by a probe is referred to as an acoustic signal.

### First Embodiment

An object information acquiring apparatus according to a first embodiment performs a plurality of photoacoustic measurements with probe scanning on a specified region of interest in an object to obtain a plurality of image data. Time-series image data are sequentially compared with each other to enable follow-up of the object. This facilitates follow-up of substance movement in the body, for example, the status of diffusion of a contrast medium in the object.

In the description below, one photoacoustic measurement or one period of photoacoustic measurement refers to a series of processes including irradiation of the object with light, probe scanning, and reception of a photoacoustic wave and executed in order to generate one image data of the region of interest. That is, a photoacoustic signal needed to generate one reconstructed image is obtained by one photoacoustic measurement or one period of photoacoustic measurement. A plurality of photoacoustic measurements is performed on the same region of interest with image reconstruction executed for each of the photoacoustic measurements to obtain time-series characteristics information. One photoacoustic measurement may be completed by one spiral movement or may include a plurality of spiral movements.

In the present invention, disturbance of a liquid surface is suppressed which may occur during one photoacoustic measurement or at the time between one photoacoustic measurement and another photoacoustic measurement as a result of a change in the acceleration of the probe (rapid acceleration, rapid deceleration, or sudden stop or turnaround). In the present invention, spiral movement and other movements are performed using a combination of curved trajectories that are curved gently to a predetermined degree or higher. The manner of curving is specified by a predetermined curvature or smaller (a predetermined radius of curvature or larger).

Alternatively, a change in the acceleration or direction of movement of the probe may be limited. Typically, the movement trajectory of the probe is controlled such that the probe moves in a pattern of small circular arcs with an aligned rotating direction.

(Photoacoustic Measurement Section)

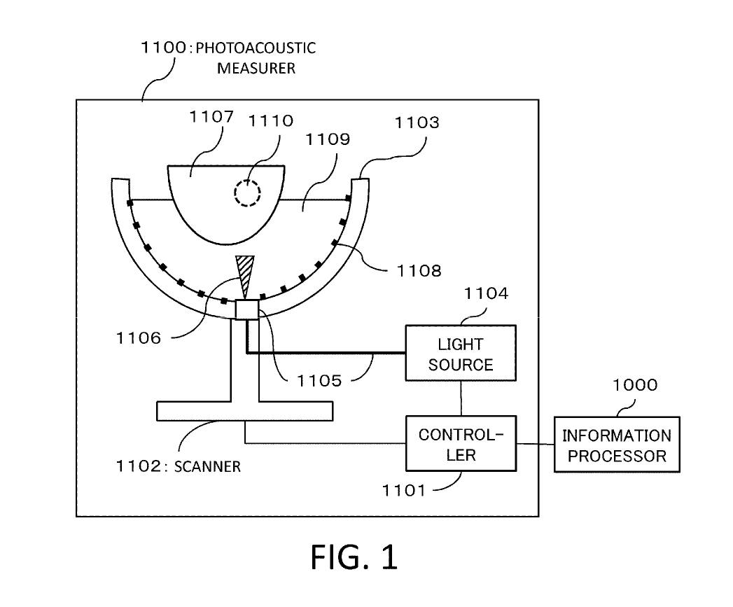

A general configuration of the object information acquiring apparatus will be described using FIG. 1. The apparatus has a photoacoustic measurer 1100 that performs photoacoustic measurement and an information processor 1000 that processes electric signals. The photoacoustic measurer 1100 transmits and receives information to and from the information processor 1000 via a controller 1101 as needed.

The controller 1101 controls blocks in the photoacoustic measurer 1100. The controller 1101 also executes an amplification process, an analog/digital conversion process, and a correction process on an electric signal output from a reception element 1108.

A scanner 1102 moves relative positions of a probe 1103 and an object 1107. The scanner 1102 may be, for example, a biaxial stage that moves the probe in an X direction and a Y direction or a triaxial stage that enables the probe to be three-dimensionally moved. The scanner 1102 moves the probe within a scan region needed to acquire characteristics information on the region of interest in accordance with instructions from the controller 1101. A movement method may be, for example, spiral movement in which the probe is revolved with respect to the object in a spiral pattern. Any movement method may be used so long as the relative positions of the probe and the object can be moved, and thus, the object may be moved.

The probe performs at least one scan on a scan region corresponding to the region of interest during one photoacoustic measurement. In order to increase the definition of a reconstructed image, the probe may perform a plurality of scans on the inside of the scan region during one photoacoustic measurement. The scan performed by the scanner 1102 includes scans during one photoacoustic measurement. The scan performed by the scanner 1102 also includes movement of the probe from an end position in one period of photoacoustic measurement to a start position in the next period. The scan performed by the scanner 1102 further includes movement of the probe between a photoacoustic measurement end position or a photoacoustic measurement start position and a home position.

The probe 1103 is a semispherical member filled with an acoustic matching member 1109. A plurality of reception elements 1108 is arranged on a semispherical surface. The object 1107 is arranged inside the probe filled with the acoustic matching member 1109. The shape of the probe is not limited to the semisphere. Any shape may be used so long as the probe can internally hold the acoustic matching member and support a plurality of reception elements 1108 such that so as to gather directional axes (directions with high reception sensitivity) of at least some of the reception elements. Examples of available shapes include a spherical crown, a part of an ellipsoid, a bowl, a dish, and a shape into which a plurality of planes or curved surfaces is combined. The shape may be a spherical zone below which a light irradiator is provided.

Instead of immersing the object 1107 directly in the acoustic matching member 1109, it is also preferable to provide a holding member that holds the object to stabilize the shape thereof. This produces effects such as improvement of measurement accuracy, a reduction in an operation time needed for the information processing apparatus to acquire the amount of light or execute a reconstruction process, and a reduction in a subject's discomfort feeling. The holding member may be a material that is highly transmissive to an acoustic wave and light. When the object is the breast, the holding member is preferably shaped like a cup or a bowl. A plurality of holding members may be prepared and replaced according to the sizes of the breasts.

The reception element 1108 preferably has high sensitivity and a wide frequency band. The reception element includes, for example, a PZT, a PVDF, a cMUT, or a Fabry-Perot interferometer. However, the present invention is not limited to such reception elements so long as the reception element can receive an acoustic wave and output an electric signal. A high-resolution region 1110 is formed near the position where the directional axes of the reception elements are gathered. The position, size, and shape of the high-resolution region may be set according to the shape of the probe or the arrangement of the elements. For example, the high-resolution region may be a region where a resolution that is half of the resolution at a central point of the semisphere can be maintained.

Alight source 1104 emits light 1106 to the object 1107. As the light source 1104, for example, a laser light source, a light emitting diode, or a flash lamp may be utilized which generates pulsed light. The light source 1104 can preferably irradiate an observation target with light with a wavelength representing to a large absorption coefficient and radiate light for a time corresponding to thermal characteristics of the object. A wavelength tunable light source is favorable.

An optical system 1105 includes various members that guide and emit the light 1106 generated by the light source 1104. The optical system 1105 includes, for example, a mirror, an optical-fiber lens, a filter, a prism, and a diffusion plate. In an illustrated example, a light emission end of the optical system 1105 is integrated with the probe 1103 and moves along with the probe 1103. Irradiation light is transmitted through the acoustic matching member 1109 and radiated to the object 1107.

The controller 1101 controls the light source 1104, the optical system 1105, and the scanner 1102 to radiate light with a timing therefor and the intensity thereof controlled to a desired position of the object. Then, the reception elements 1108 receive a photoacoustic wave under the control of the controller 1101. An analog electric signal resulting from the photoacoustic wave is converted, through amplification and analog-digital conversion, into a photoacoustic signal, which is sent to the information processor 1000 along with conditions present at the time of acquisition of the signal.

The conditions present at the time of acquisition of the photoacoustic signal include information on the reception elements such as the positions of the reception elements on a reception surface of the probe 1103 and the sensitivity and directionality of the reception elements. The conditions also include parameters for the light source and the optical system and light amount information. The conditions further include control information on movement of the probe 1103, positional information on the probe 1103 at each point in time, and information that allows determination of a position where each electric signal has been acquired. To obtain one image data, a plurality of photoacoustic waves may be acquired at the same position and integrated or summed and averaged during one photoacoustic measurement to improve an SN ratio. In this case, each of a plurality of signal intensities or the calculated information may be sent to the information processor 1000.

The acoustic matching member 1109 occupies the space between the object 1107 and the probe 1103 to acoustically match the object 1107 and the probe 1103 with each other. A material for the acoustic matching member 1109 is desirably a liquid that has an acoustic impedance close to the acoustic impedances of the object 1107 and the reception elements 1108 and allows pulsed light to pass through. As the material, for example, water, castor oil, or gel is used. However, the present invention is not limited to these materials so long as the material enables acoustic matching.

(Information Processing Section)

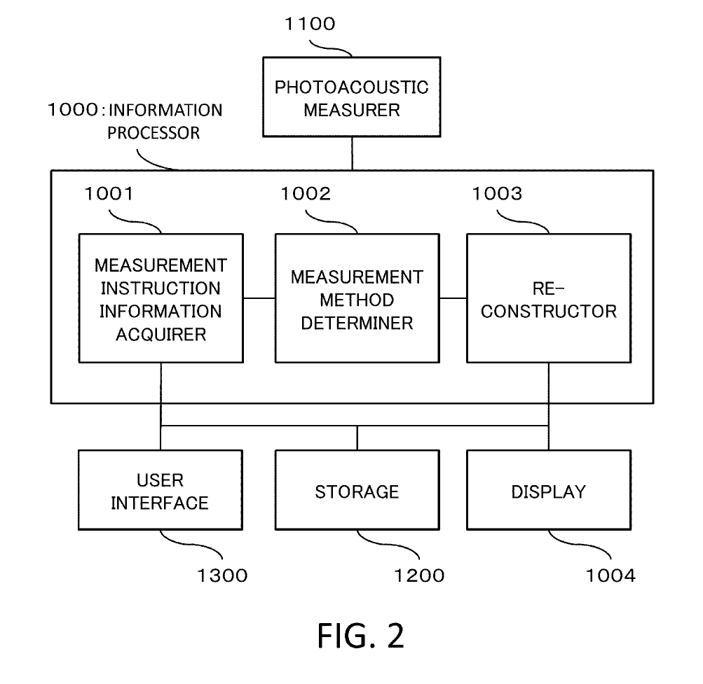

FIG. 2 depicts functional blocks of the information processor 1000. The information processor 1000 acquires a measurement instruction from a user (a physician or a laboratory technician) and converts the measurement instruction into control information for the photoacoustic measurer 1100. The control information is generated to meet the user's desire for image quality, the number of measurements, measurement intervals, and the like and to enable a liquid surface of the acoustic matching member to be restrained from being disturbed. The information processor 1000 also generates characteristics information on the interior of the object using a photoacoustic signal obtained by photoacoustic measurement, and for example, saves or displays the characteristics information.

The information processor 1000 includes a measurement instruction information acquirer 1001, a measurement method determiner 1002, and a reconstructor 1003. The information processor 1000 can communicate with a display 1004, the photoacoustic measurer 1100, and a storage 1200. The display 1004 may be provided separately from or integrally with the information processor 1000.

The measurement instruction information acquirer 1001 receives measurement instructions from the user via a user interface 1300 such as a mouse, a keyboard, or a touch panel. An example of the measurement instruction is a range specification for the region of interest to be reconstructed. The range specification is performed using a method such as coordinate specification, input via a touch interface, or selection from prescribed values specific to the apparatus. For example, the method may involve selection from a plurality of preset three-dimensional regions and fine-tuning of the selected region, specification via the mouse or the touch panel with reference to images taken with a camera (not depicted in the drawings), or input of coordinate values.

The contents of instructions other than the range specification include specification, in the case of a plurality of photoacoustic measurements, of the number of photoacoustic measurements and the intervals between the photoacoustic measurements, the image quality of one image data obtained during one photoacoustic measurement, and set parameters for a photoacoustic measurement function. When consecutive photoacoustic measurements are unwanted, the number of photoacoustic measurements is zero. The measurement instruction information acquirer 1001 transmits acquired measurement instruction information to the measurement method determiner 1002. The measurement instructions may be stored in the storage 1200 such that the user can select from the measurement instructions.

The measurement method determiner 1002 determines a measurement method for the photoacoustic measurer 1100 based on the acquired measurement instruction information. The measurement method includes information needed to determine control parameters provided to the light source 1104, the optical system 1105, the scanner 1102, and the like by the controller 1101. That is, the controller 1101 performs control according to the measurement method to acquire a photoacoustic signal needed to reconstruct an image of the region of interest. The measurement method determiner 1002 transmits the determined measurement method to the photoacoustic measurer 1100 and the reconstructor 1003.

The reconstructor 1003 receives the photoacoustic signal from the photoacoustic measurer 1100. If conditions present at the time of acquisition of the signal are available, the reconstructor 1003 receives the conditions. The reconstructor 1003 generates volume data for each reconstruction unit (each voxel or pixel) in the region of interest using the photoacoustic signal, the signal acquisition conditions and information on the measurement method. The reconstruction may be performed utilizing various known methods such as universal back-projection, filtered back projection, and sequential reconstruction.

The reconstructor 1003 stores generated image data in the storage 1200 in a desired data format. The image data may be stored along with the measurement method information, the signal acquisition conditions, the measurement instruction information or the like, as needed. For example, volume data representing the region of interest in terms of the signal intensity for each voxel is saved in combination with information on the reconstructed image. At this time, “Digital Imaging and COmmunications in Medicine” (DICOM) format may be utilized which is a standard for medical images. Information specific to the present invention, for example, the number of photoacoustic measurements and the accuracy of images can be stored utilizing private tags in the DICOM format.

Any storage may be used as the storage 1200 so long as the storage communicates with an information processing apparatus or is built into the information processing apparatus so as to be able to store information. For example, a magnetic disk, an optical disc, or a semiconductor memory may be used.

The reconstructor 1003 may transmit the generated characteristics information to the display 1004 to allow the display 1004 to display the image. Examples of a method for displaying an image obtained by one photoacoustic measurement may include a method of displaying a three-dimensional region of interest in a plurality of tomographic views, maximum intensity projection, and volume rendering. When a plurality of photoacoustic measurements is performed on the same region of interest to genera a plurality of images in a time series, the images may be displayed in line or in a moving-image form in which the image is changed at regular time intervals. Moreover, the image of the object may be displayed along with information on the conditions for the photoacoustic measurement or on the measurement method, object information obtained using a modality other than PAT, past images of the same subject, text information with annotations, or the like.

As the display 1004, a liquid-crystal display, a plasma display, a CRT display, or the like may be utilized. However, the present invention is not limited to these displays.

In the present embodiment, the photoacoustic measurer 1100 is separated from the information processor 1000. However, the photoacoustic measurer 1100 and the information processor 1000 may be integrated together. Some of the blocks of the photoacoustic measurer 1100 and some of the blocks of the information processor 1000 may be arranged outside the photoacoustic measurer 1100 and the information processor 1000, respectively. The information processor 1000 may be an information processing apparatus having a processing circuit such as a CPU, a storage apparatus, a user interface, and a display apparatus and enabling input/output and calculation of information in accordance with an OS, a control program, or an application program. A possible such apparatus is a workstation, a PC, or the like. The blocks included in the information processor 1000 may be formed using different circuits or formed as functional modules of a program that allows the CPU to execute an object information acquiring method. The reconstructor corresponds to a processor of the present invention. Furthermore, the controller 1101 is suitably an information processing apparatus.

(Process Flow of Measurement Instruction)

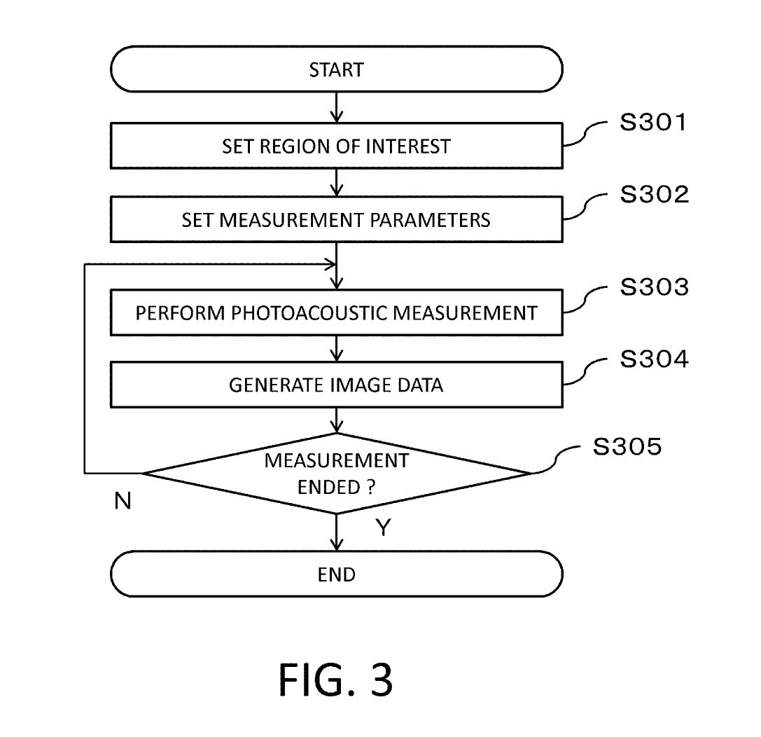

A specific process procedure will be described using a flowchart. FIG. 3 is a flowchart illustrating a process in which the user measure s the subject's breast as an object. This flow starts at a point in time when the breast is set at a predetermined position. In the apparatus in FIG. 1, the subject lying on the subject's stomach with the breast hanging downward and installed inside the acoustic matching member occupying the inside of the probe 1103.

In step S301, the user uses one of the above-described methods to set, in the breast, a three-dimensional region of interest to be reconstructed.

In step S302, the user sets various measurement parameters. The parameters include the type of a scan trajectory (spiral or circle), conditions for image quality such as a scan pitch and the number of measurement points, and reconstruction conditions. The present invention allows specification of parameters such as the number of photoacoustic measurements and the intervals between photoacoustic measurements in the case of a plurality of photoacoustic measurements, a method for controlling the probe at the time between photoacoustic measurements, and a method for controlling the probe between trajectories of spiral movements. The number of spiral movements during one photoacoustic measurement may be specified by the user or determined by the apparatus according to the needed image quality. The process in the present step may be automatically set according to the contents of the input in step S301.

In step S303, when the user gives an instruction to start measurement, the information processor 1000 and the photoacoustic measurer 1100 perform photoacoustic measurement in an interlocking manner. Thus, a photoacoustic signal is acquired which is needed to generate one reconstructed image. At this time, the measurement method determiner 1002 generates control information based on the default set items and contents of the user's instruction acquired by the measurement instruction information acquirer 1001. The contents of the control include control of the light source and light irradiation and setting of a scan region corresponding to the region of interest. The contents also include a movement trajectory of the probe corresponding to the desired measurement accuracy, the moving speed of the probe, the number of photoacoustic measurements, the density of measurement positions, and control information for the probe needed to achieve the movement trajectory, the moving speed, the number of photoacoustic measurements, and the density of measurement positions. The contents further include control information on information processing such as correction. These parameters may be selected from prescribed values according to the user's specification.

Subsequently, in step S304, the information processor 1000 uses the photoacoustic signal to generate image data indicative of characteristics information on the inside of the region of interest. This process may be collectively executed after all the photoacoustic measurements end.

In step S305, when the end of a specified number of photoacoustic measurements is determined, the present flow ends. A determination condition may be, besides the number of measurements, whether the quality of the image data obtained in S304 meets a predetermined criterion or whether the user has given an instruction to end measurements. After the flow is completed, the image data may be saved or the image is displayed on the display.

(Scan Trajectory of Probe)

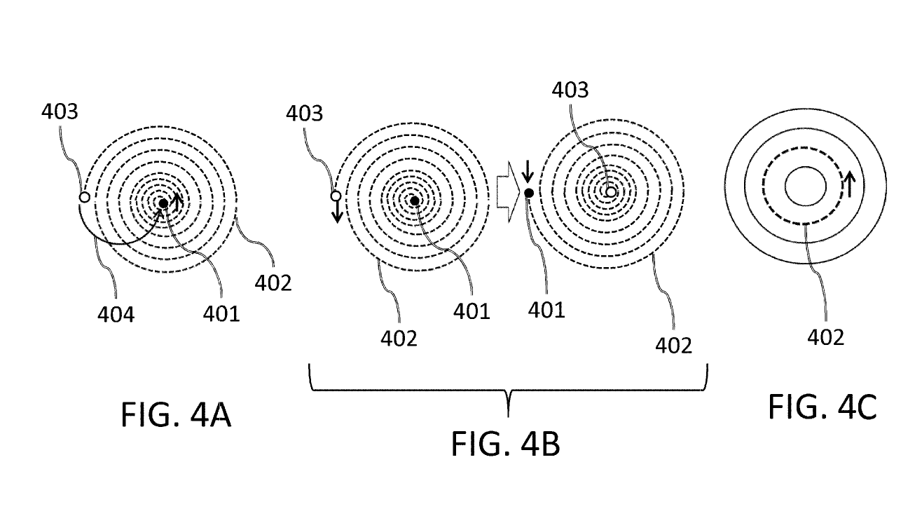

Scans of the probe 1103 during one photoacoustic measurement will be described using FIGS. 4A, 4B, and 4C. An example will be described where the scanner 1102 spirally moves the probe 1103 in a scan region corresponding to the region of interest. In FIGS. 4A, 4B, and 4C, the trajectory of a central point of a semispherical probe as viewed from above in the vertical direction (from the subject side) is depicted by a dashed line. Although not depicted here, the probe 1103 may rotate.

FIG. 4A depicts the trajectory of the central point during spiral scans. A spiral trajectory depicted by reference numeral 402 extends from a start position 401 to an end position 403. Electric signals needed for reconstruction are obtained from photoacoustic waves acquired at some positions on the trajectory. A connection trajectory depicted by reference numeral 404 is used if the same region of interest is repeatedly measured during one photoacoustic measurement or the same region of interest is consecutively measured during a plurality of photoacoustic measurements. The former repeated measurements are performed in order to improve an SN ratio, whereas the latter consecutive measurements are performed in creating moving images or a time lapse movie for follow-up.

The connection trajectory 404 joints the end position 403 to the start position 401 for the next photoacoustic measurement, with a curved trajectory (typically, a circular arc) that rotates in the same direction as the rotating direction of the trajectory 402. This prevents the probe 1103 from performing a sudden turnaround, restraining the liquid surface from being disturbed. This is because the probe moves in a direction along the flow of the acoustic matching member resulting from the spiral motion. At this time, control is preferably performed so as to prevent the probe 1103 from changing rapidly in speed. A connection trajectory with the same rotating direction may be used even when the probe moves from the outside to the inside of the spiral.

In FIG. 4A, the start point for the second photoacoustic measurement is the same as the start point for the preceding photoacoustic measurement. However, the start position may be displaced for each photoacoustic measurement to allow a wide region of interest to be measured. Spirals with different sizes, different measurement densities, and different positions of measurement points may be used for the respective photoacoustic measurements. An increased number of measurement points allow an artifact reduction effect to be exerted. Instead of moving from the center to the outside of the spiral as depicted in FIG. 4A, the probe may move from the outside to the inside of the spiral. For a constant flow of the acoustic matching member, a consecutive measurement scheme is more preferable than a step and repeat scheme.

FIG. 4B illustrates a scan method different from the scan method in FIG. 4A. The end position 403 in the left of FIG. 4B depicts the same place as the start position 401 in the right of FIG. 4B. The left of FIG. 4B represents the preceding spiral movement in the same region of interest, whereas the right of FIG. 4B represents the subsequent spiral movement in the same region of interest. As depicted in the left of FIG. 4B, the probe first moves on the trajectory 402 from the start position 401 located at the center of the preceding spiral to the end position 403 located outside. Then, as depicted in the right of FIG. 4B, the probe 1103 moves from the outside to the inside of the subsequent spiral. In this case, each of the movements may be considered to be one photoacoustic measurement or the two movements may be collectively considered to be a single photoacoustic measurement. This method also allows the probe to be continuously moved without going against the flow of the acoustic matching member, enabling a reduction in the disturbance of the liquid surface. Moreover, a trajectory having a larger radius of curvature than the connection trajectory 404 in FIG. 4A allows a plurality of spirals to be connected together. This is very effective for restraining the liquid surface from being disturbed.

FIG. 4C depicts the trajectory 402 along which the central position of the probe 1103 moves on a circle. The size of the circle can be optionally specified according to the size of the region of interest. For example, one rotation on the same circle corresponds to one period of photoacoustic measurement. When the probe moves between a plurality of circular trajectories with different radii, such trajectories as to prevent the acoustic matching member from being disturbed are used. In any of FIGS. 4A to 4C, the rotating direction of the probe may be either clockwise or counterclockwise.

The trajectory of the probe may be shaped like an ellipse according to the shape of the region of interest or a spiral the outermost periphery of which can be approximated by an ellipse. However, in this case, a curve with a relatively large curvature is formed near the focus of the ellipse, and thus, ellipticity and the moving speed are set such that a fluctuation of the liquid surface at the curve falls within a predetermined range. Furthermore, the present invention is not limited to the case where all the trajectories are curved. For example, the present invention may be utilized in a case where a shape resulting from division of a circle into two semicircles and connection of the semicircles with two lines. In this case, turnaround occurs near the junction between the line and the semicircle and is dealt with by moving speed control.

In some apparatuses, the home position of the probe 1103 is predetermined. In this case, at the start of photoacoustic measurement, the probe 1103 moves from the home position to the start position 401. After the end of the photoacoustic measurement, the probe 1103 moves from the end position 403 to the home position. The liquid surface may be disturbed at the start of the photoacoustic measurement depending on the movement trajectory or the moving speed. Thus, the probe is controlled such that, when the probe moves from the home position to the start position or moves from the end position to the home position, a curved trajectory is also formed along the rotating direction of a spiral trajectory or a circular trajectory.

(Process Flow of Photoacoustic Measurement)

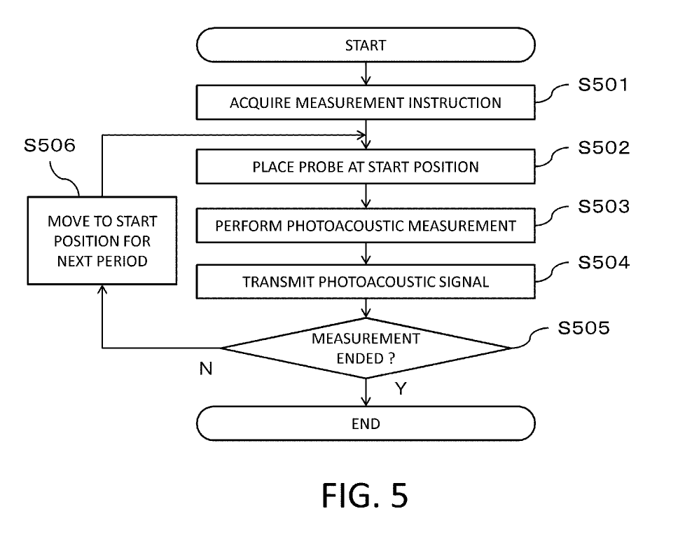

FIG. 5 is a flowchart illustrating a process procedure executed by the photoacoustic measurer 1100 upon receiving a measurement instruction. The present flow starts when the measurement instruction is received from the information processor 1000.

In step S501, the controller 1101 receives the measurement method from the information processor 1000. In step S502, the controller 1101 controls the light source 1104, the optical system 1105, and the scanner 1102 to complete preparation for the start of photoacoustic measurement. For example, when the probe 1103 is at the home position, the scanner 1102 moves the probe 1103 to the start position 401. At this time, the probe 1103 preferably makes curved motion along the curve during the subsequent photoacoustic measurement. However, during the first period of consecutive measurements, the measurement may be started when a time enough to settle the disturbance of the liquid surface passes after the movement.

In step S503, the controller 1101 synchronizes the moving speed and the movement trajectory of the probe and the position of and a timing for light irradiation, and receives a photoacoustic wave in the scan region to generate a photoacoustic signal. The present step is executed until acoustic-wave reception corresponding to one photoacoustic measurement is completed. In the present embodiment, such a trajectory as depicted in FIG. 4A is formed.

As described above, one photoacoustic measurement is a concept including various measurement schemes allowing one image data to be generated. For example, one photoacoustic measurement may include a plurality of spiral scans in the same scan region. Alternatively, one photoacoustic measurement may be allowed to cover a wide scan region by combining a plurality of spirals together.

In step S504, the photoacoustic signal is transmitted from the controller 1101 to the information processor 1000. Transmitted information may include positional information on the elements corresponding to each signal, information on reception time, and information on acquisition conditions for the photoacoustic wave. Alternatively, an average value or a representative value for a plurality of signals received at the same position may be transmitted.

In step S505, the controller 1101 determines whether the number of measurements specified in the measurement method has been reached. When the number of measurements has been reached, the process is ended. Besides the number of measurements, the user's input of a stop instruction may be used as an end condition. On the other hand, when the number of measurements has not been reached, the process proceeds to step S506 to move the probe 1103 to the next photoacoustic measurement position.

In step S506, the controller 1101 moves the probe 1103 to the start position 401 for the next photoacoustic measurement. At this time, the probe is moved in the same curve direction as that of the probe movement during the preceding photoacoustic measurement. Thus, curved motion is made in the same direction as that of the flow of the acoustic matching member. Furthermore, the radius of curvature of the movement is preferably maximized to form a gentle curve. The speed of the movement may be made the same as or close to the speed of the photoacoustic measurement. The above-described series of processes is repeated a specified number of times to complete the present process.

In the present flow, the moving direction of the probe coincides or accords with the rotating direction of the acoustic matching member at the start and end of measurement, at the time between consecutive photoacoustic measurements, and at the time between a plurality of spiral movements. Thus, the liquid surface can be restrained from being disturbed. Moreover, changes in the moving speed of the probe are slowed to allow the liquid surface to be more effectively undisturbed. As a result, the space between the object or the holding member and the reception elements is prevented from being mixed with air layers or air bubbles, whereby good image quality can be obtained.

If the scanner 1102 has a component that comes into contact with the acoustic matching member (such as an arm that allows the probe 1103 to be rotated), the component is preferably allowed to make circular motion in the same rotating direction as that of the acoustic matching member. The same can be said for components other than the probe 1103 (for example, an optical sensor).

(Information Processing Flow)

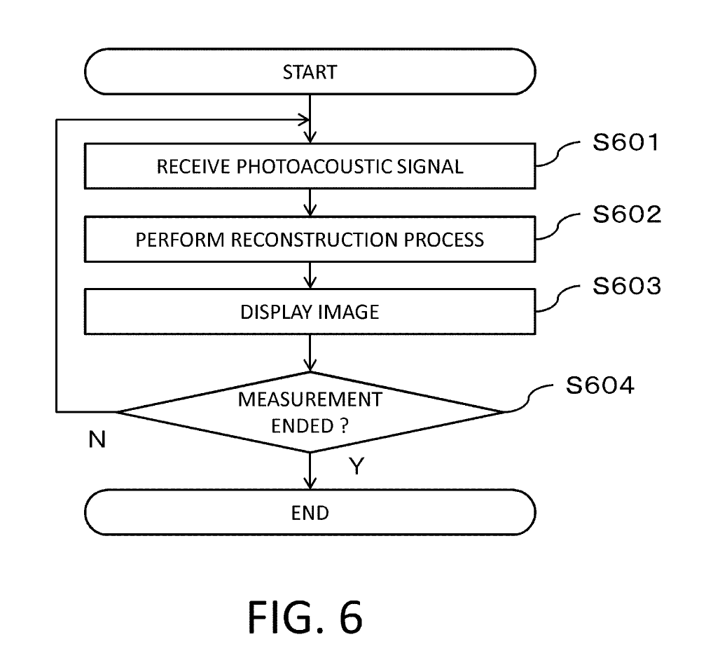

A flowchart in FIG. 6 illustrates a process procedure executed by the information processor 1000. The present flow starts when the information processor 1000 receives a photoacoustic signal from the photoacoustic measurer 1100.

In step S601, the information processor 1000 receives a photoacoustic signal and attached information from the photoacoustic measurer 1100. In step S602, the reconstructor 1003 performs image reconstruction. Thus, the user's desired information is obtained such as an initial sound pressure distribution, an absorption coefficient distribution, a substance concentration distribution, or an oxygen saturation distribution inside the region of interest. In step S603, the reconstructor 1003 transmits image data to the display 1004 to allow the display 1004 to display the image data in the user's desired manner. In addition to or as an alternative to display of the image data, storage of the image data in the storage 1200 may be performed.

In step S604, whether or not the photoacoustic measurement has ended is determined. When the photoacoustic measurement has not ended, the process returns to S601 to receive a photoacoustic signal resulting from the next measurement. When a scheme in which a moving image is created after reception of all the measurement results is executed instead of a sequential display scheme according to the progress of the photoacoustic measurement, the process in S604 is not needed.

An object image displayed in the present flow results from a photoacoustic wave acquired with the disturbance of the acoustic matching member reduced. Thus, the present flow allows provision of good images restrained from being affected by air layers or bubbles.

In the above description, the photoacoustic measurer 1100 transmits a photoacoustic signal to the information processor 1000 after one period of photoacoustic measurement ends. However, the photoacoustic signal may sequentially be transmitted to the information processor 1000 during one period of photoacoustic measurement. In this case, the information processor 1000 also executes a sequential reconstruction process, effectively reducing process intervals and improving image quality.

### Second Embodiment

In a second embodiment, photoacoustic measurement that is a combination of two types of spiral-movement control methods is executed as depicted in FIG. 4B. Thus, compared to the first embodiment, the second embodiment reduces changes in the moving direction or the angular speed of the probe 1103 that may cause the acoustic matching member to be disturbed. Moreover, the second embodiment involves no movement time between photoacoustic measurements, enabling consecutive and quick photoacoustic measurements. Even in this case, the rotating direction remains the same during the time between photoacoustic measurements, restraining the liquid surface from being disturbed.

An apparatus configuration in the second embodiment is similar to the apparatus configuration in the first embodiment. A process flow in the second embodiment is different from the process flow in the first embodiment in the contents of the setting in S302 in FIG. 3 and the processes in S502, S503, S506, and the like in FIG. 5. The probe movement in the left of FIG. 4B (from inside to outside) and the subsequent probe movement in the right of FIG. 4B (from outside to inside) are hereinafter treated as one set. That is, one photoacoustic measurement in the present embodiment refers to generation of a photoacoustic signal in one set. However, a plurality of sets may be combined into one photoacoustic measurement.

In particular, in the present embodiment, the above-described one set of photoacoustic signal generation is performed in S503 in FIG. 5. In FIG. 4A, a circular-arc-shaped connection trajectory 404 is used to join a plurality of spirals together and has a relatively small radius of curvature and a sharp curve. On the other hand, in FIG. 4B, spirals are smoothly connected together, leading to a relatively large radius of curvature and relatively gradual changes in angle. Thus, the liquid surface is more appropriately restrained from being disturbed, improving image quality.

### Third Embodiment

In a third embodiment, a case will be described where the probe moves on the same circle as in FIG. 4C. This reduces changes in the moving direction or the angular speed of the probe 1103 that may cause the acoustic matching member to be disturbed. The need for the movement time between periods is also eliminated, enabling consecutive measurements.

The present embodiment is different from the above-described embodiments in the control of the probe. That is, the probe 1103 moves on a circle with a given size set according to the region of interest. The photoacoustic measurer 1100 performs photoacoustic measurement based on the scan range of the probe 1103 determined by the measurement method determiner 1002 as depicted in FIG. 4C.

For a circular trajectory as in the present embodiment, the start position and the end position can be set at any positions. In the present embodiment, one lap around the circle corresponds to one photoacoustic measurement. However, a trajectory with a plurality of laps around the circle may correspond to one photoacoustic measurement. Alternatively, a part of the circle may correspond to one photoacoustic measurement. Alternatively, a trajectory along a plurality of circles with different radii may correspond to one photoacoustic measurement. In that case, the trajectory is such that the distance of the movement between the circles is not the shortest (that is, the normal direction of a tangent to a circle) and such that the angle of movement is prevented from being steep. The present embodiment allows the flow of the acoustic matching member to be further restrained from being disturbed.

(Variation)

In the above-described embodiments, the region of interest is a three-dimensional region. However, the region of interest may be a two-dimensional plane, and a plurality of images may be consecutively generated. For example, any one section in a three-dimensional region may be utilized as a two-dimensional plane.

In a plurality of photoacoustic measurements, the consecutive measurements may be executed with the central position of a spiral trajectory (the start position 401 in FIG. 4A) slightly shifted between periods. This results in a large number of photoacoustic signals with different measurement positions, allowing reconstructed images with reduced artifact to be consecutively generated. Furthermore, a wide region of interest can be dealt with.

A combination of a plurality of trajectories may be used during one photoacoustic measurement or during one series of measurements. Furthermore, conditions for light irradiation may be switched for each period. For example, light with different wavelengths is used for the respective plurality of photoacoustic measurements to enable the concentrations of oxyhemoglobin and deoxyhemoglobin to be determined, allowing an oxygen saturation distribution to be acquired.

The object of the present invention can be accomplished when the probe moves such that the acceleration of the probe and a change in the moving direction of the probe fall within respective predetermined ranges. The object of the present invention can also be accomplished when the acceleration of the probe and the change in the moving direction of the probe fall within such ranges as to bring a change in the liquid surface of the acoustic matching member resulting from the movement of the probe within a predetermined range.

As described above, the present invention allows suppression of disturbance of the liquid surface of the acoustic matching member resulting from movement of the probe near the interface of the object (or the holding member). As a result, the accuracy of photoacoustic measurement is restrained from decreasing, allowing images with reduced artifact to be acquired. In particular, when the same region of interest is consecutively measured a plurality of times, the liquid surface is restrained from being disturbed at the time between the measurements. Thus, moving images or time lapse images with high quality can be acquired. This is advantageous for diagnosis and follow-up using a contrast medium or the like.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-013245, filed on Jan. 27, 2015, which is hereby incorporated by reference herein in its entirety.

What is claimed is:

1. An object information acquiring apparatus comprising:
a light source;
a plurality of elements each receiving a photoacoustic wave generated in a region of interest of an object irradiated with light from the light source, and outputting an electric signal;
a probe that supports the plurality of elements such that directional axes of at least some of the plurality of elements are gathered, and that holds acoustic matching material acoustically matching the object with the plurality of elements;
a scanner that moves the probe;
a controller that controls the light source, the plurality of elements, and the scanner; and
a processor that generates image data on the region of interest using electric signals output by the plurality of elements upon receiving the photoacoustic wave at each position to which the probe is moved by the scanner,
wherein the controller performs, on the same region of interest, a plurality of photoacoustic measurements in which the probe is moved along a curved trajectory in a scan region corresponding to the region of interest, with each of the plurality of elements receiving the photoacoustic wave, and moves the probe in the same rotating direction as a rotating direction of the curved trajectory at a time between the photoacoustic measurements, and
the processor generates a plurality of the image data based on the plurality of photoacoustic measurements in the same region of interest.

2. The object information acquiring apparatus according to claim 1, wherein the controller controls the scanner such that the probe performs spiral movement.

3. The object information acquiring apparatus according to claim 2, wherein the controller controls the scanner such that trajectories of a plurality of the spiral movements in the same region of interest are connected together with a curved connection trajectory with the same rotating direction as the rotating direction of the trajectories of the spiral movements.

4. The object information acquiring apparatus according to claim 2, wherein the controller controls the scanner such that subsequent spiral movement in the same region of interest starts at an end position of preceding spiral movement in the same region of interest.

5. The object information acquiring apparatus according to claim 2, wherein the controller controls the scanner such that each of the photoacoustic measurements includes a plurality of the spiral movements.

6. The object information acquiring apparatus according to claim 2, wherein the controller controls the scanner such that the probe performs a plurality of the spiral movements with a start position shifted for each of the spiral movements.

7. The object information acquiring apparatus according to claim 1, wherein the controller controls the scanner such that the probe moves on a circle.

8. The object information acquiring apparatus according to claim 1, wherein a home position is set for the probe, and
the controller controls the scanner such that movement of the probe from the home position to a start position at a start of the photoacoustic measurement is along a curve with the same rotating direction as the rotating direction of the curved trajectory in the photoacoustic measurement.

9. The object information acquiring apparatus according to claim 1, wherein a home position is set for the probe, and
the controller controls the scanner such that movement of the probe from an end position to the home position after an end of the photoacoustic measurement is along a curve with the same rotating direction as the rotating direction of the curved trajectory in the photoacoustic measurement.

10. The object information acquiring apparatus according to claim 1, wherein the controller controls the scanner such that the probe moves such that an acceleration of the probe and a change in a moving direction of the probe fall within respective predetermined ranges.

11. The object information acquiring apparatus according to claim 1, wherein the controller controls the scanner such that an acceleration of the probe and a change in a moving direction of the probe fall within such ranges as to bring a change in a liquid surface of the acoustic matching member resulting from the movement of the probe within a predetermined range.

12. An object information acquiring apparatus comprising:
a light source;
a probe supporting a plurality of elements each receiving a photoacoustic wave, the probe configured to hold acoustic matching material that provides acoustic matching between the elements and the object;
a scanner that moves the probe;
a controller that controls the scanner in a manner that the probe moves from an end position of a photoacoustic measurement and a start position of a subsequent photoacoustic measurement along a curved trajectory.
